(19) United States
(12) Patent Application Publication
(10) Pub. No.: US 2021/0282751 A1
(43) Pub. Date: Sep. 16, 2021

(54) PORTABLE 3D VOLUMETRIC ULTRASOUND IMAGE GUIDANCE SYSTEM FOR PRONE PARTIAL BREAST IRRADIATION

(71) Applicant: THE BOARD OF REGENTS OF THE UNIVERSITY OF TEXAS SYSTEM, Austin, TX (US)

(72) Inventor: Xuejun GU, (US)

(21) Appl. No.: 16/321,786

(22) PCT Filed: Jul. 28, 2017
(86) PCT No.: PCT/US17/44383

§ 371 Date: Jan. 29, 2019

Related U.S. Application Data
(60) Provisional application No. 62/368,918, filed on Jul. 29, 2016.

(51) Int. Cl. A61B 8/08; A61B 8/00; A61N 5/10

(52) U.S. Cl. CPC A61B 8/5253; A61B 8/406; A61B 8/0825; A61N 2005/1058; A61B 8/483; A61B 8/4281; A61N 5/10; A61B 8/466

(57) ABSTRACT

Embodiments of devices, apparatuses, and methods for a portable 3D ultrasound image-guided system, which may provide high quality volumetric images of breast tissue in treatment planning and patient positioning during radiation therapy treatment courses.

### CROSS-REFERENCE TO RELATED APPLICATIONS

This application claims priority to U.S. Provisional Patent Application No. 62/368,918, filed Jul. 29, 2016, which application is incorporated by reference in its entirety.

### BACKGROUND

### 1. Field of the Invention

The present invention relates generally to breast imaging and treatment, and more particularly, but not by way of limitation, to devices, apparatuses, and methods for providing three dimensional (3D) volumetric ultrasound (US) imaging for use with prone partial breast irradiation treatments.

### 2. Description of Related Art

In stereotactic body radiation treatment (SBRT) regimens, a specially designed coordinate system may be used to determine a precise location of treatment sites such as cancerous tumors. The treatments sites may then be treated with a focused high dose of radiation or a few fractionated radiation treatments. Additionally, multiple radiation beam angles may be used to safely deliver high doses of radiation to the treatment sites with very sharp dose gradient outside the treatment site and into the surrounding healthy tissue. Due to the ability to deliver a potent dose of radiation to the treatment site with minimal delivery to healthy tissue, a cure rate of a tumor may be increased and collateral damage to healthy tissue may be decreased.

One use of SBRT is for treatment of breast cancer. Various breast imaging systems have been developed for breast cancer diagnosis, including x-ray based tomosynthesis, on-board cone-beam computed tomosynthesis (CBCT), fiducial marker tracking, breast magnetic resonance imaging (MRI), and an automatic breast ultrasound system (ABUS). In some prior embodiments, CT and/or MRI images may be provided to determine the treatment areas for the radiation application. However, this may provide some challenges, such as a difficulty providing an accurate picture for target localization and difficulty maintaining proper patient positioning for treatment.

Currently, in partial breast irradiation treatment, there is no effective image-guide system available to localize treatment targets. Fiducial markers as treatment target surrogates can only indirectly indicate treatment target position. Small errors in fiducial markers' position may incur large errors of treatment target localization. Moreover, implanting fiducial markers is an invasive procedure and implanted fiducial markers are prone to migration in breasts. X-ray based CBCT is not an effective image modality for breasts, as it provides very low soft tissue contrast. It is also impossible to use the current linear accelerator (LINAC) based on-board CBCT images to localize treatment target volume in breasts. While MRI is an effective soft tissue imaging modality, integrating MRI into LINAC requires enormous tasks. There are some research developments of MRI-LINAC; however, these specialized machines will not be widely available due to numerous technique barriers and high costs. Additionally, during breast treatment, there is no monitoring of treatment effectiveness during the course of radiation treatment and often no treatment plan adaptation if no visible changes are observed on the treated area.

### SUMMARY

This disclosure includes embodiments of devices, apparatuses, and methods for providing three dimensional (3D) volumetric ultrasound imaging for use with prone partial breast irradiation treatments.

Some embodiments of the present apparatuses comprise an ultrasound imaging apparatus comprising a reservoir comprising a body defining an interior compartment having a central axis and configured to contain a liquid, and an opening in fluid communication with the compartment, the reservoir configured to receive a portion of a patient's breast into the compartment through the opening; a cradle coupled to the body of the reservoir such that the cradle is configured to rotate relative to a fixed support and around the central axis of the reservoir, the cradle configured to be coupled to an ultrasound transceiver such that the ultrasound transceiver is fixed in at least one degree of freedom relative to the cradle.

In some embodiments, the cradle is movable relative to the body, and where the reservoir is configured to remain stationary as the cradle rotates around the central axis. In some embodiments, the cradle and the reservoir form a unitary piece, where the unitary piece is configured to rotate around the central axis. In some embodiments, the reservoir is configured to rotate around the central axis. In some embodiments, the reservoir and the cradle are configured to synchronously rotate around the central axis. In some embodiments, the cradle is configured to pause at one or more intervals during a rotation around the central axis. In some embodiments, the one or more intervals comprise one or more angular increments around the central axis. In some embodiments, the one or more angular increments may include a plurality of 5-degree increments. In some embodiments, the cradle is configured to permit the ultrasound transceiver to translate parallel to the vertical axis while the ultrasound transceiver is coupled to the cradle. In some embodiments, the cradle is coupled to the body of the reservoir such that the cradle is translatable relative to the body parallel to the vertical axis. In some embodiments, the cradle is configured to pause at one or more intervals during a translation parallel the vertical axis.

In some embodiments, the ultrasound transceiver is configured to take one or more images at each of a plurality of angular intervals as the cradle rotates around the central axis. In some embodiments, the ultrasound transceiver is further configured to take one or more images at each of a plurality of planes that are spaced along and perpendicular to the vertical axis. In some embodiments, the reservoir is configured to receive a breast mold surrounding the portion of the patient's breast. In some embodiments, the ultrasound imaging apparatus further comprises a mount coupled to the cradle such that the cradle can rotate relative to the mount, a portion of the mount configured to remain stationary as the cradle rotates, the stationary portion configured to be accessible from or through an upper side of the cradle. In some embodiments, an upper surface of the stationary portion may comprise one or more protrusions or recess. In some embodiments, the reservoir is configured to receive a breast mold surrounding the portion of the patient's breast. In some embodiments, the stationary portion is configured to secure a breast mold such that the breast mold is prevented from rotating around the central axis. In some embodiments, the ultrasound imaging apparatus further comprises a breast mold comprising a cup portion defining a recess, and a rim portion coupled to the cup portion around the recess, the cup portion being configured to contain the breast tissue within the recess, and the rim portion being configured to secure the breast mold to the stationary portion. In some embodiments, the cup portion of the breast mold is configured to extend into the reservoir through the opening of the reservoir with the portion of the patient's breast disposed in the recess of the cup portion. In some embodiments, the breast mold comprises a material configured to permit ultrasound imaging through the material. In some embodiments, the breast mold is specific to a particular patient. In some embodiments, the shape of the breast mold corresponds to a model generated from one or more medical images of the portion of the patient's breast of the patient. In some embodiments, one or more step motors are configured to rotate the cradle about the central axis and/or move the cradle along the vertical axis of the reservoir. In some embodiments, the one or more step motors are further configured to receive control signals from at least one processor, the control signals configured to control a rate of rotation of the cradle about the central axis and/or a rate of movement of the cradle along the vertical axis of the reservoir.

Some embodiments of the present apparatuses comprise a breast tissue immobilizing apparatus comprising a breast mold comprising a cup portion defining a recess, and a rim portion coupled to the cup portion around the recess, the breast mold comprising a material configured to permit ultrasound imaging through the cup portion; where the breast mold is specific to a particular patient. In some embodiments, the cup portion is configured to contain breast tissue within the recess, and the rim portion is configured to secure the breast mold to an ultrasound imaging apparatus. In some embodiments, the shape of the breast mold corresponds to a model generated from one or more medical images of a breast of the patient.

Some embodiments of the present methods comprise a method of imaging breast tissue, the method comprising inserting the breast into a reservoir; immobilizing a breast of a patient; filling at least a portion of the reservoir with a liquid; rotating an ultrasound transceiver around a periphery of the reservoir; and imaging the breast with the ultrasound transceiver. In some embodiments, the method further comprises pausing the rotation of the ultrasound transceiver at one or more intervals. In some embodiments, the method further comprises creating one or more three dimensional (3D) images of the breast based on one or more images taken at the one or more intervals. In some embodiments, the method further comprises determining a location of one or more treatment areas of the breast based on the one or more 3D images. In some embodiments, the method further comprises applying radiation to the one or more treatment areas. In some embodiments, the method further comprises moving the ultrasound transceiver along a vertical axis of the reservoir. In some embodiments, the method further comprises pausing the movement of the ultrasound transceiver at one or more intervals. In some embodiments, the method further comprises imaging the breast at one or more planes along the vertical axis of the reservoir. In some embodiments, immobilizing the breast further comprises maintaining the breast within a customized breast mold. In some embodiments, the shape of the customized breast mold corresponds to a model generated from one or more medical images of a breast of the patient. In some embodiments, creating one or more 3D images of the breast further comprises receiving, by at least one processor, the one or more images taken at the one or more intervals; and performing, by the at least one processor, a super-compounding process on the one or more images. In some embodiments of the present methods, a method of treating breast tissue comprises applying radiation to a treatment volume in a breast of a patient, where the radiation is directed to the treatment volume in accordance with a 3D model generated from a plurality of ultrasound images of the breast; and where the breast remains immobilized between the time at which the ultrasound images are obtained and the radiation is applied to the treatment volume.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present devices, apparatuses, kits, and methods, the term “substantially” may be substituted with “within [a percentage] of what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device, or structure that is configured in a certain way is configured in at least that way, but it can also be configured in other ways than those specifically described.

Any embodiment of any of the present devices, apparatuses, kits, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiments depicted in the figures.

FIG. 1 depicts views of a treatment images of a prior art SBRT.

FIG. 2 depicts a SBRT system according to an embodiment of the disclosure.

FIG. 3 depicts an ultrasound imaging device according to an embodiment of the disclosure.

FIG. 4 depicts a perspective view of an ultrasound imaging device according to an embodiment of the disclosure.

FIG. 5 depicts a comparison between a single ultrasound image and a super-compounded ultrasound image that may be acquired according to an embodiment of the present disclosure.

FIG. 6 depicts various views of a super-compounded 3D ultrasound image that may be acquired according to an embodiment of the present disclosure.

FIG. 7 depicts a comparison between a CT image, an MRI image, and a super-compounded ultrasound image that may be acquired according to an embodiment of the present disclosure.

FIG. 8 depicts a view of a transducer trajectory for acquiring image data according to an embodiment of the present disclosure.

FIG. 9 depicts a view illustrating a technique for capturing image data according to an embodiment of the present disclosure.

FIG. 10 depicts a flow diagram of a method for imaging a breast with an ultrasound imaging device according to an embodiment of the present disclosure.

FIG. 11 depicts a flow diagram of a method for treating breast tissue according to an embodiment of the present disclosure.

### DESCRIPTION OF ILLUSTRATIVE EMBODIMENTS

Compared to tomosynthesis and CBCT, the developed portable 3D US imaging system has advantages in providing higher contrast soft tissue images and an easier treatment target localization. Breast MRI scanning can provide good soft tissue images, but it is much more expensive than the developed 3D US system. Moreover, due to complexity of MRI, integrating MRI as an on-board imaging system faces enormous technique barriers. Commercially available 3D US breast imaging systems can provide high quality volumetric breast images at a reasonable cost; however, US scans with these systems often require breast compression, which is not suitable for radiotherapy treatment's target localization and positioning.

The system disclosed herein is the first 3D US image-guided system developed for partial breast irradiation. The breast holder is a unique design, which utilizes patient CT images to create patient-specific breast holders. The designed breast holder is an effective immobilization device for stabilizing prone breast during US scanning and radiation delivery. The rotational water container is also a special design, which simplifies transducer and water tank coupling issue and imaging calibration. The super-compounding technique is special, which can suppress image noise with an effective angular compounding process. The system disclosed herein offers key advantages over existing systems. No existing 3D US imaging system is designed for guiding breast radiotherapy treatment and has a non-touch US scanning technique. The patient-specific breast holder is also a special design, which assists non-touch US scanning and immobilizing/positioning breast during radiation treatment.

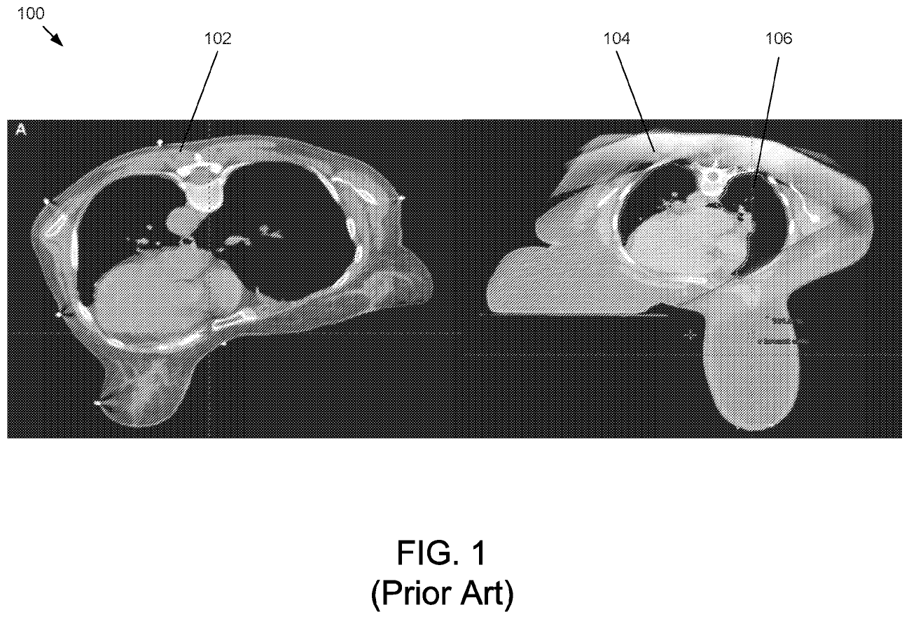

Prone partial breast irradiation may require positioning precision for accurate radiation dose delivery. However, accurate dose delivery is often challenging due to deformation of the breast and shrinkage of the lumpectomy cavity during imaging. Due to the characteristics of soft tissue, it may be difficult to reproduce breast treatment positions. This is shown in FIG. 1, which depicts views 100 of a treatment images of a prior art embodiment of SBRT. Image 102 depicts a breast image taken at one point in time while image 104 depicts a breast image taken at another point in time. Image 104 shows how the shape of the breast is deformed as compared to image 102 and also how the lumpectomy cavity 106 has shrunk compared to image 102. Because each of the breast images may have a different shape and configuration, it may be difficult to determine appropriate treatment sites for radiation dose delivery.

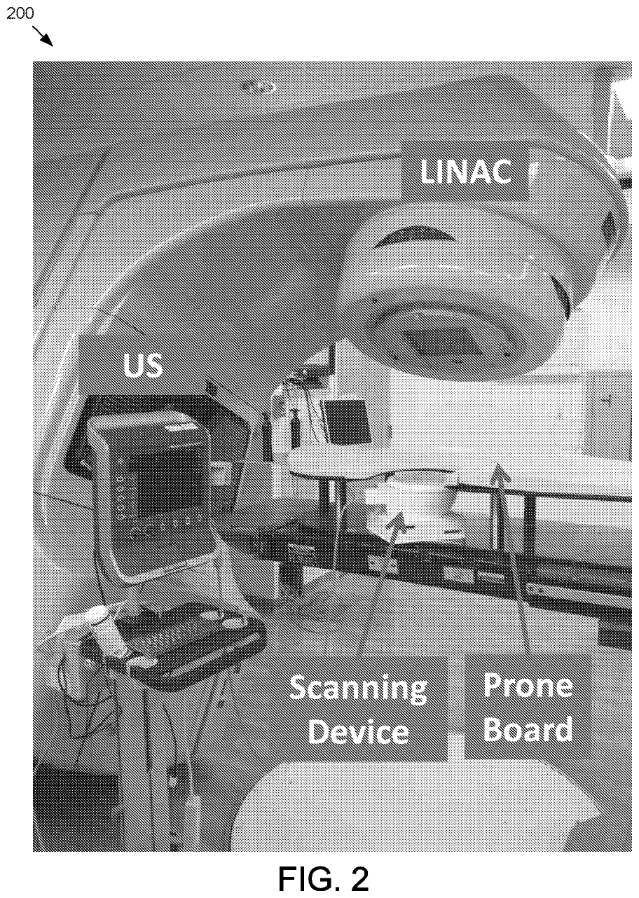

FIG. 2 depicts a SBRT system 200 according to an embodiment of the disclosure. The developed portable 3D US image-guided system solves several critical problems of the prior art partial breast irradiation treatment. 1) Breast immobilization: The patient-specific breast holder of the current embodiments can help reproduce breast shape and position. 2) Treatment target localization: The developed portable 3D US imaging-guidance system may be able to provide high quality volumetric images of soft breast tissue for treatment target localization with very low costs. Meanwhile, its portable characteristics may allow for an easy integration with the current radiotherapy treatment workflow. 3) Treatment progress monitoring: The developed 3D volumetric US imaging system provides a non-invasive treatment effectiveness monitoring. Fractional volumetric ultrasound images can provide longitudinal information of the treatment target/whole breast anatomy and the tissue elastic property along treatment courses, allowing for treatment plan adaptation and a possible radio genomic analysis.

In the embodiment shown, the 3D ultrasound image-guided system may comprise an ultrasound image acquisition hardware system and a 3D ultrasound image reconstruction software system. In the embodiment shown, the image acquisition system may be compatible with a clinical adopted prone breast couch/board. In the embodiment shown, the main components of system may include a portable US scanning/imaging device, which can be placed underneath the prone breast couch during US image acquisition and moved away during radiation treatment delivery.

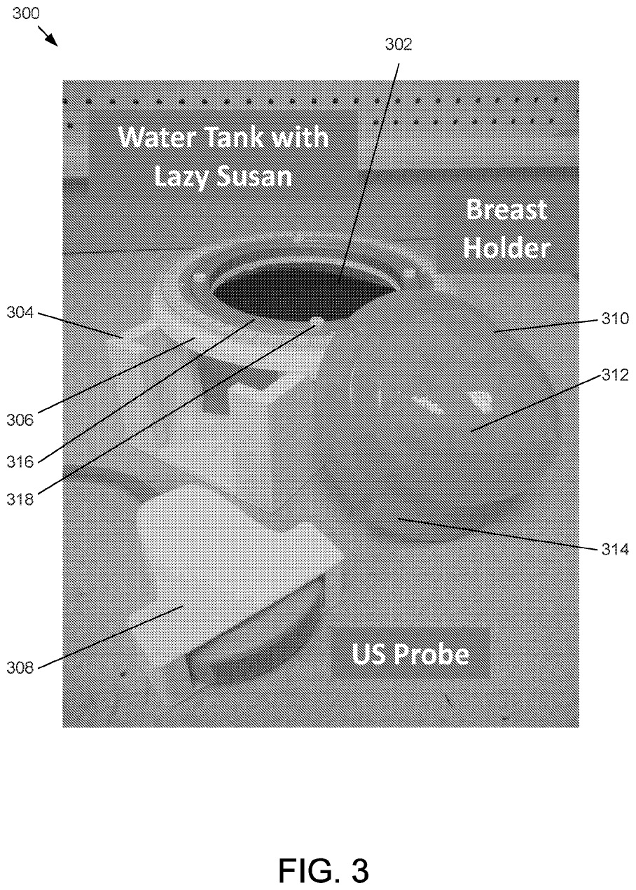

FIG. 3 depicts an ultrasound imaging device 300 according to an embodiment of the disclosure. In the embodiment shown, the device 300 may include a leak-proof water container/reservoir 302. In some embodiments, the device 300 may be configured to allow the reservoir 302 to be moved up and/or down in a vertical direction to accommodate the position of the prone breast couch. In some embodiments, a cradle 304 may be coupled to the reservoir 302. In some embodiments, the reservoir may be integrated with one or more rotational devices 306 (e.g., Lazy Susans). In some embodiments, a rotational device 306 may be attached to the bottom of the water container, on the top of the container around the opening of the container, or both. The rotational devices 306 may enable the water reservoir 302 to rotate without perturbing the immersed breast. In some embodiments, a US transceiver/transducer/probe 308 may be attached to the side of the reservoir 302 via cradle 304 having a specially designed slot. In some embodiments, the US transceiver 308 may be separated from the water reservoir 302 by a sealed membrane.

In some embodiments, a personalized breast holder/mold 310 may be provided, which may immobilize the breast during US scanning and radiation treatment delivery. In some embodiments, the breast mold 310 may include a cup portion 312 and a rim portion 314. The breast mold 310 may be developed based on a model generated from medical images of a particular patient. For example, various CT, MRI, and/or ultrasound images or the like maybe used to generate a model of a patient's breast. A customized breast mold 310 may then be created from the model. The customized breast mold 310 may be created from a material that allows ultrasound images to be taken through the material. In some embodiments, a stationary member or ring 316 may be provided at the opening of the reservoir 302. The stationary ring 316 may be configured to secure the breast mold 310 to the scanning apparatus when the breast mold 310 is protruding into the reservoir. In some embodiments, the stationary ring 316 may have one or more stationary protrusions or pins 318 that may be used to secure the breast mold 310. For example, the breast mold illustrated in FIG. 3 includes a plurality of notches that may be seated or interfaced with the one or more stationary protrusions or pins 318 to secure the breast mold 310. In other embodiments, the stationary ring 316 may use a different technique to secure the breast mold 310. For example, one or more pints may be located on the breast mold 310 and the stationary ring 316 may include notches configured to interface with the one or more pints on the breast mold 310 to secure the breast mold 310. As another example, the outer edge of the ring 314 of the breast mold 310 may include a first threaded portion and the stationary ring 316 may include a second threaded portion, and the breast mold 310 may be secured to the stationary ring 316 by rotating the US imaging device 300 to cause the second threaded portion to engage the first threaded portion. It is noted that the particular mechanisms for securing the position of the breast with respect to the US imaging device 300 have been disclosed herein for purposes of illustration, rather than by way of limitation, and that embodiments of the present disclosure may utilize other techniques to secure the position of the breast mold 310 with respect to the US imaging device 300, as will be apparent to a person of ordinary skill in the art upon reading the present disclosure.

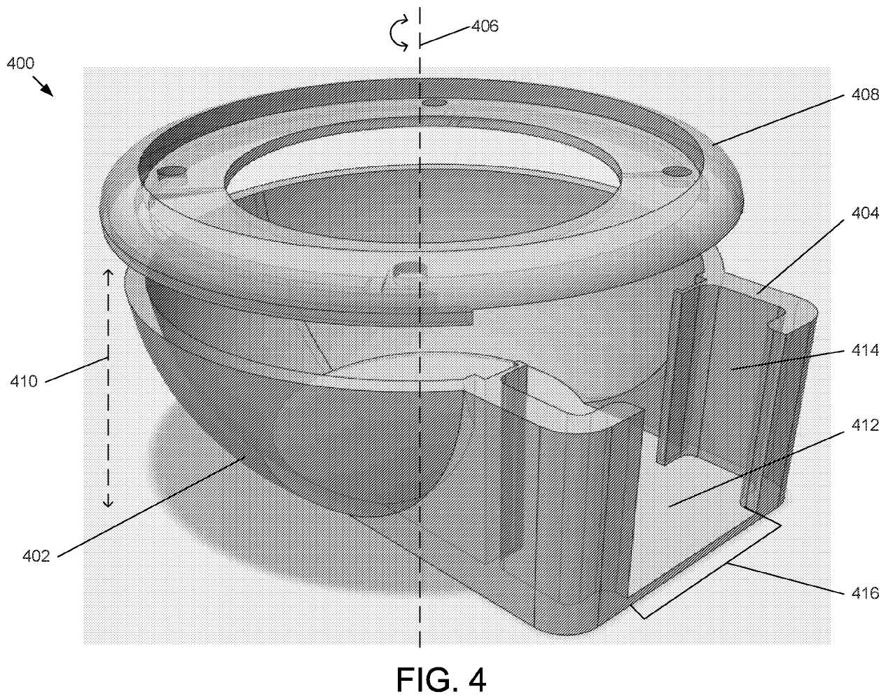

FIG. 4 depicts a perspective view 400 of an ultrasound imaging device according to an embodiment of the disclosure. In the embodiment shown, the reservoir 402 may be coupled to the cradle 404 to form a unitary piece. The unitary piece may be coupled to one or more rotational devices in enable the unitary piece to rotate around a central axis 406 of the reservoir 402. In some embodiments, the cradle 404 may be separate from the reservoir 402 but may be configured to rotate around the central axis 406 of the reservoir 402. In some embodiments, the reservoir 402 may be stationary while the cradle 404 rotates around the central axis 406 of the reservoir 402. In other embodiments, the reservoir 402 may rotate along with the cradle 404 in synchrony or at different speeds. Stationary ring 408 may be disposed at the opening of the reservoir 402 and configured to secure the breast mold 310 into the reservoir 402. In some embodiments, a mount (not shown) may be provided that may be coupled to the cradle 404. In some embodiments, the mount may remain stationary as the cradle 404 rotates. In some embodiments, the cradle 404 may be able to move along a vertical axis 410 of the reservoir 302. In some embodiments, the cradle 404 may comprise a recess 412, one or more vertical grooves 414, and one or more lateral openings 416 that may be configured to removably attach the US transceiver 308 to the cradle 404. The US transceiver 308 may be inserted into the top of recess 412 and may be held stationary by the one or more vertical grooves 414. In some embodiments, the one or more lateral openings 416 may be configured to enable a US scanning portion of the US transceiver 308 to face toward the reservoir 402 and/or may be configured to enable a power supply portion of the US transceiver 308 to protrude out of the recess 412 and away from the reservoir 402 and the cradle 404. As the cradle 404 rotates and/or moves vertically, the US transceiver 308 may move with it in the same manner in order to take the ultrasound images.

In some embodiments, the ultrasound imaging device may enable a rotational US scan of the breast in a coronal plane. The rotation and/or movements of the cradle 304, 404 may enable the US transceiver 308 to record ultrasound images at different positions of the breast. The cradle 304, 404 may be configured to rotate continuously or intermittently, pausing at certain intervals. In some embodiments, the US transceiver 308 may record an image at 5 degree increments as the cradle rotates. In some embodiments, the US transceiver 308 may also record images at different planes as the cradle 404 moves up and/or down the vertical axis 410 of the reservoir 302, 402.

In some embodiments, a US rotational scan control system may be provided. In some embodiments, a dedicated scanning control computer with at least one microcontroller/processor may control the US probe's rotation and vertical elevation with one or more step motors (not shown). In some embodiments, one or more step motors may be configured to rotate the cradle 304, 404 around a central axis of the reservoir 302, 402 while the same one or more step motors or different one or more step motors may be configured to move the cradle 304, 404 along the vertical axis 410 of the reservoir 302, 402.

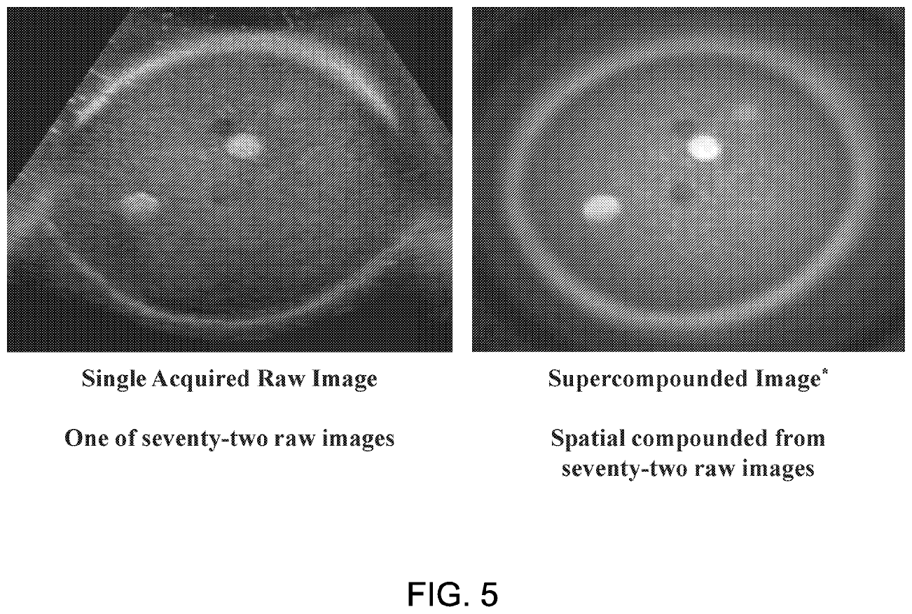

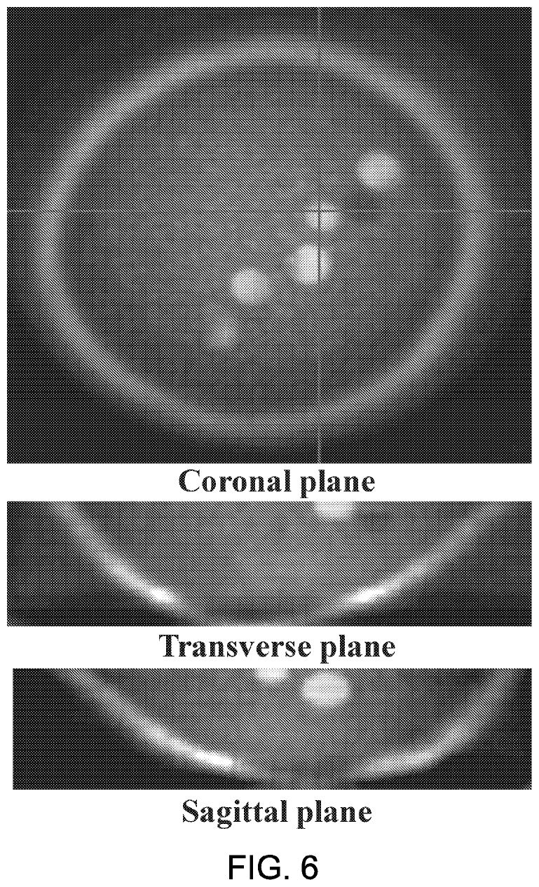

In the developed software platform, volumetric US reconstruction may be realized with a super-compounded technique. The super-compounding technique may comprise an angular spatial compounding method based on a two-dimensional spin-average concept that may be used to reduce angular dependent artifacts and provide a spatially invariant point spread function in all directions. The reconstructed uniform-resolution and artifact suppressed 3D volumetric image may have a higher image quality compared to conventional two dimensional (2D) US images. FIG. 5 depicts a comparison between a single ultrasound image and a super-compounded ultrasound image that may be acquired according to an embodiment of the present disclosure. In some embodiments, the single ultrasound images may be taken by the US transceiver at various intervals as the cradle rotates around the breast and/or moves along the vertical axis of the reservoir. As can be seen, the resolution of the super-compounded image may provide a higher resolution that may more easily delineate healthy breast tissue from objects that may be detected within the breast tissue such as markers, tumors, or the like. FIG. 6 depicts various views of a super-compounded 3D ultrasound image that may be acquired according to an embodiment of the present disclosure. In the embodiment shown, the 3D image is shown from the coronal plane, the transverse plane, and the sagittal plane but other planes may also be examined. These super-compounded images may be created because the breast may be imaged from various angles and/or planes while remaining immobilized in a stable position. The embodiments shown, the images were taken during a breast phantom study of a volume of 13×11×7 cm. The exemplary image sizes were 640×480 pixels per inch with a space between slices of 2 mm.

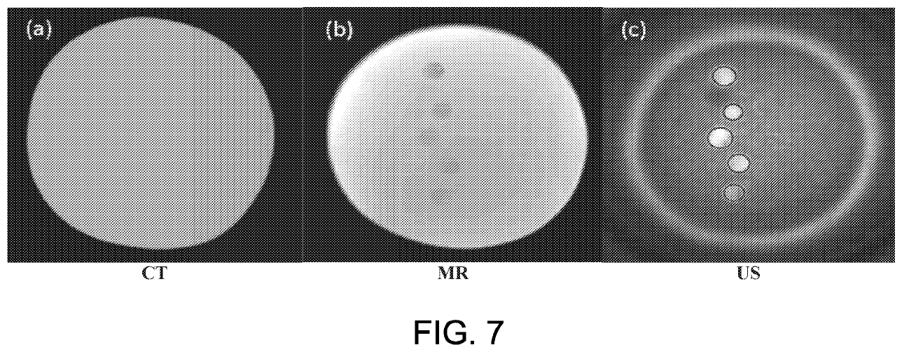

FIG. 7 depicts a comparison between a CT image, an MRI image, and a super-compounded ultrasound image that may be acquired according to an embodiment of the present disclosure. In the embodiment shown, the images show results of a breast phantom study, where a breast phantom was scanned using the different image modalities of CT, breast MRI, and US. In the CT image (a), there is no showing of any objects that may be contained within the breast tissue that may delineate desired radiation treatment areas. In the MRI image (b), five reflection objects are detected but the resolution is low. In the US image (c), the five reflection objects may be identified on a cross section of US reconstructed images that match the objects shown in MRI image. However, the US images provide a higher resolution that may assist in delineating the objects from the rest of the breast tissue. The below tables illustrate the differences in the detected diameter of the reflective objects (TABLE 1) and the detected distance of the reflective objects (TABLE 2) between US images and MRI images.

TABLE 1
Diameter of reflective objects (mm)

| Obj. | US | MR | Diff. |
|---|---|---|---|
| 1 | 10.2 | 7.6 | 2.6 |
| 2 | 8.5 | 6.8 | 1.7 |
| 3 | 11.0 | 8.2 | 2.8 |
| 4 | 9.7 | 8.7 | 1.0 |
| 5 | 8.9 | 6.7 | 2.2 |

TABLE 2
Distance between adjacent reflective objects (mm)

| Dist. | US | MR | Diff |
|---|---|---|---|
| 1-2 | 19.4 | 18.9 | 0.5 |
| 2-3 | 14.9 | 14.0 | 0.9 |
| 3-4 | 15.8 | 15.1 | 0.7 |
| 4-5 | 15.8 | 15.0 | 0.8 |

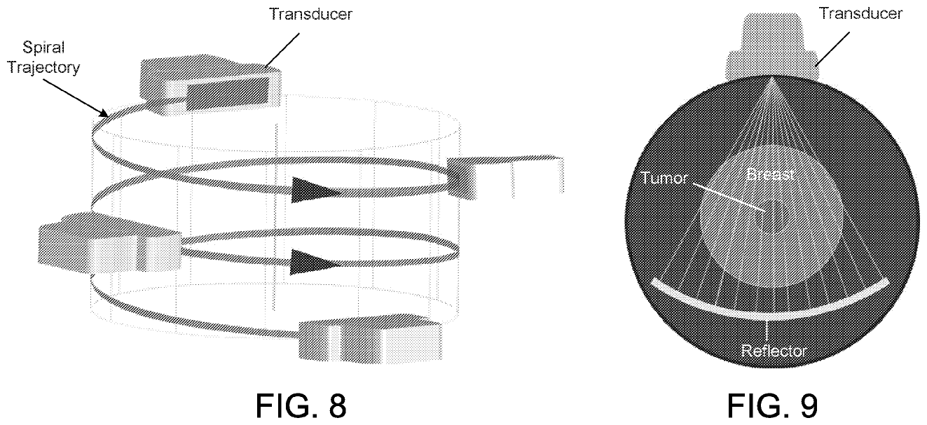

FIG. 8 depicts a view of a transducer trajectory for acquiring image data according to an embodiment of the present disclosure. As described above with respect to FIG. 4, during US scanning of a breast using a US imaging device configured according to embodiments of the present disclosure (e.g., the US imaging device 300 of FIG. 3 and/or the imaging device illustrated in FIG. 4) a US transceiver (e.g., the US transceiver 308 of FIG. 3) may be rotated around the breast to obtain US image data. As shown in FIG. 8, in an embodiment, the US transceiver may be rotated around the breast such that a transducer of the US transceiver has a spiral trajectory. By using a spiral trajectory, image data may be acquired for the entire breast.

In some embodiments, a guide mechanism may be provided to guide the rotation of the US transceiver. For example, a cradle of the US imaging device (e.g., the cradle 304 of FIG. 3 and/or the cradle 404 of FIG. 4) may include one or more channels configured to interface with one or more grooves on an exterior surface of a water reservoir of the US imaging device (e.g., the water reservoir 302 of FIG. 3). The grooves and channels may be configured to guide rotation of the US transceiver so that the US transducer is rotated in a manner that provides the spiral trajectory illustrated in FIG. 8. Further, by using the grooves/channels to control the trajectory of the US transducer, the rotation may be performed smoothly because the operator of the US imaging device does not have to manually control the position of the US transceiver during the rotation. It is noted that although the description above describes including channels on the cradle and grooves on the exterior surface of the water reservoir, embodiments may utilize configurations where the grooves are provided on the cradle and the channels are provided on the exterior surface of the water reservoir. Further, it is noted that the use of channels and grooves to control the trajectory of the transducer has been described for purposes of illustration, rather than by way of limitation, and that other techniques for controlling the trajectory of the transducer during imaging of a breast may be used in accordance with embodiments of the present disclosure. In some embodiments, no mechanism for controlling the trajectory of the transducer may be provided, and the user may manually control the rotation and trajectory of the transducer during an imaging process. Additionally, it is noted that use of a spiral trajectory, as shown in FIG. 8, has been provided for purposes of illustration, rather than by way of limitation, and that embodiments of the present disclosure may utilize rotation techniques that provide other trajectories, such as a series of circular trajectories, in accordance with the present disclosure.

FIG. 9 depicts a view illustrating a configuration of a US imaging device for capturing image data according to an embodiment of the present disclosure. As shown in FIG. 9, a sound reflector may be installed on an interior wall of a reservoir (e.g., the reservoir 302 of FIG. 3) of an US imaging device (e.g., the imaging device 300 illustrated in FIG. 3 and/or the US imaging device illustrated in FIG. 4) in accordance with an embodiment of the present disclosure. As the US transducer of the US transceiver is rotated about the breast during scanning, the sound reflector may reflect the US signals, and the reflections may be used to produce and/or enhance images generated based on image data captured during operation of the US imaging device. For example, in an embodiment, sinogram data may be compiled based on time delay measurements collected at one or more transceiver positions during the imaging process, and the collected sinogram data may be fed into a filter back projection algorithm to reconstruct sound speed tomography images. In another embodiment, the sinogram data may be representative of sound speed attenuation measurements compiled at each transceiver position over 360, and the collected sinogram data may be used to attenuation tomography images may be reconstructed by feeding the sinogram data into a filter back projection algorithm. In an embodiment, the reconstructed sound speed and attenuation images may be used for B-mode image correction, enabling the image generated using the US imaging device of the present disclosure to approach the quality as quantitative and multichromatic 3D images generated using more complex and costly systems. In an embodiment, automatic image analysis may be performed using artificial Intelligence technologies. For example, deep learning techniques based on quantitative US images of reflection, attenuation and sound speed may be used to enable the imaging devices of embodiments to provide automatic breast cancer screening and diagnosis.

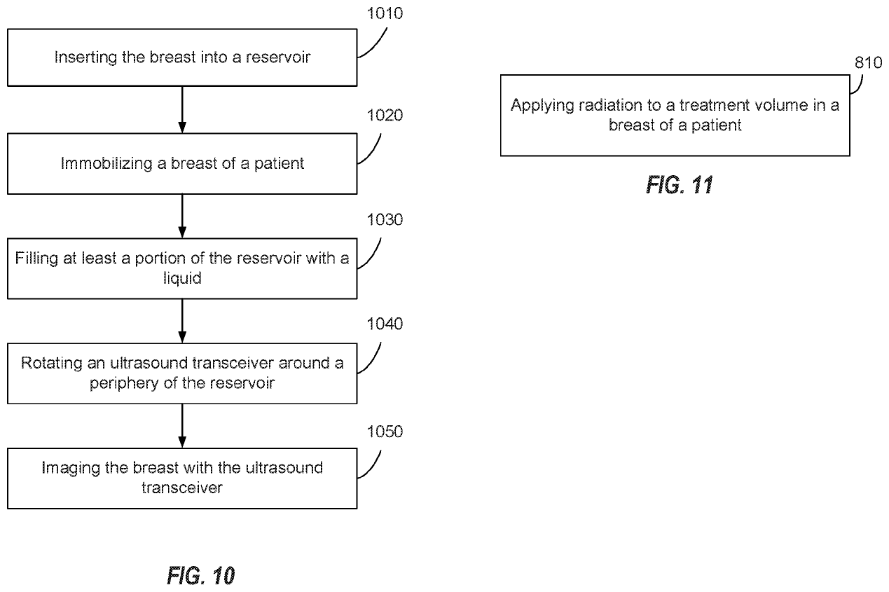

Referring to FIG. 10, a flow diagram of a method for imaging a breast with an ultrasound imaging device according to an embodiment of the present disclosure is shown as a method 1000. In an embodiment, the method 1000 may be performed using one or more of the US imaging devices described above with respect to FIGS. 3, 4, 8, and 9. As shown in FIG. 10, the method 1000 includes, at step 1010, inserting a breast into a reservoir. In an embodiment, the reservoir may be the reservoir 302 of FIG. 3. At step 1020, the method 1000 includes immobilizing a breast of a patient. In an embodiment, the breast may be immobilized using a breast mold configured to interface with a portion of the US imaging device, as described above with reference to FIG. 3. At step 1030, the method 1000 include filling at least a portion of the reservoir with a liquid. In an embodiment, the reservoir may be filled with liquid that is contained in a sealed environment, and step 1030 may be omitted. At step 1040, the method 1000 includes rotating an ultrasound transceiver around a periphery of the reservoir. In an embodiment, the transceiver may be rotated around the periphery manually. In an embodiment, rotation of the transceiver may be guided by one or more guide mechanisms, as described above with respect to FIG. 8. At step 1050, the method 1000 includes imaging the breast with the ultrasound transceiver. In an embodiment, the imaging of the breast may be performed in the manner described above with reference to FIGS. 3-9.

Referring to FIG. 11, a flow diagram of a method for treating breast tissue according to an embodiment of the present disclosure is shown as a method 1100. As shown in FIG. 11, the method 1100 includes, at step 1110, applying radiation to a treatment volume in a breast of a patient. In an embodiment, the radiation may be directed to the treatment volume in accordance with a 3D model generated from a plurality of ultrasound images of the breast. In an embodiment, the plurality of ultrasound images of the breast may be generated according to the method 1000 using a US imaging device configured according to embodiments of the present disclosure, such as the imaging devices illustrated and described with reference to FIGS. 3-9. In an embodiment, the breast remains immobilized between the time at which the ultrasound images are obtained and the radiation is applied to the treatment volume.

With the in-room super-compounded 3D volumetric ultrasound image guidance system described in the embodiments herein, it may be possible to generate a high quality volumetric US image with high contrast on soft tissue. This may enable a better field of view of treatment regions of interest and enable a better delineation between the treatment target and surrounding structures. A better understanding of the treatment progress may be available by the ability to observe planning target volume (PT.) volume changes. The system may provide better patient positioning by using patient geometrics instead of indirect alignment. The system may provide a cost-effective solution for prone breast SBRT image guidance and a breast scanning technique that does not compress or deform the breast tissue.

Using this system, a patient can receive a 3D US scan before each treatment fraction for treatment target positioning. This image-guided system may improve the radiation beam delivery precision, reduce radiation treatment margin and consequently reduce radiation treatment toxicity. Moreover, this 3D US image-guided system may open a new approach for radiation treatment monitoring and adaptation. With fractional 3D volumetric US images, it may also be possible to perform a radio-genomic study to predict the outcome of treatment.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices, apparatuses, kits, and methods are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An ultrasound imaging apparatus comprising:
a reservoir comprising a body defining an interior compartment having a central axis and configured to contain a liquid, and an opening in fluid communication with the compartment, the reservoir configured to receive a portion of a patient's breast into the compartment through the opening;
a cradle coupled to the body of the reservoir such that the cradle is configured to rotate relative to a fixed support and around the central axis of the reservoir, the cradle configured to be coupled to an ultrasound transceiver such that the ultrasound transceiver is fixed in at least one degree of freedom relative to the cradle.

2. The ultrasound imaging apparatus of claim 1, where the cradle is movable relative to the body, and where the reservoir is configured to remain stationary as the cradle rotates around the central axis.

3-9. (canceled)

10. The ultrasound imaging apparatus of claim 1, where the cradle is coupled to the body of the reservoir such that the cradle is translatable relative to the body parallel to the vertical axis.

11. The ultrasound imaging apparatus of claim 10, where the cradle is configured to pause at one or more intervals during a translation parallel the vertical axis.

12-13. (canceled)

14. The ultrasound imaging apparatus of claim 1, where the reservoir is configured to receive a breast mold surrounding the portion of the patient's breast.

15. The ultrasound imaging apparatus of claim 1, further comprising:
a mount coupled to the cradle such that the cradle can rotate relative to the mount, a portion of the mount configured to remain stationary as the cradle rotates, the stationary portion configured to be accessible from or through an upper side of the cradle.

16-18. (canceled)

18. The ultrasound imaging apparatus of claim 15, where the stationary portion is configured to secure a breast mold such that the breast mold is prevented from rotating around the central axis.

19. The ultrasound imaging apparatus of claim 18, further comprising:
a breast mold comprising a cup portion defining a recess, and a rim portion coupled to the cup portion around the recess, the cup portion being configured to contain the breast tissue within the recess, and the rim portion being configured to secure the breast mold to the stationary portion.

20. The ultrasound imaging apparatus of claim 19, where the cup portion of the breast mold is configured to extend into the reservoir through the opening of the reservoir with the portion of the patient's breast disposed in the recess of the cup portion.

21. (canceled)

22. The ultrasound imaging apparatus of claim 17, where the breast mold is specific to a particular patient.

23. (canceled)

24. The ultrasound imaging apparatus of claim 1, further comprising:
one or more step motors configured to rotate the cradle about the central axis and/or move the cradle along the vertical axis of the reservoir.

25. (canceled)

26. The ultrasound imaging apparatus of any of claims 1-25, wherein the reservoir comprises a reflector.

27. A breast tissue immobilizing apparatus comprising:
a breast mold comprising a cup portion defining a recess, and a rim portion coupled to the cup portion around the recess, the breast mold comprising a material configured to permit ultrasound imaging through the cup portion;
where the breast mold is specific to a particular patient.

28. The apparatus of claim 27, where the cup portion is configured to contain breast tissue within the recess, and the rim portion is configured to secure the breast mold to an ultrasound imaging apparatus.

29. (canceled)

30. The ultrasound imaging apparatus of claim 27, wherein the reservoir comprises a reflector.

31. A method of imaging breast tissue, the method comprising:
inserting the breast into a reservoir;
immobilizing a breast of a patient;
filling at least a portion of the reservoir with a liquid;
rotating an ultrasound transceiver around a periphery of the reservoir; and
imaging the breast with the ultrasound transceiver.

32. (canceled)

33. The method of claim 31, further comprising:
creating one or more three dimensional (3D) images of the breast based on one or more images taken at the one or more intervals.

34-35. (canceled)

36. The method of claim 31, further comprising:
moving the ultrasound transceiver along a vertical axis of the reservoir.

37-38. (canceled)

39. The method of claim 31, where immobilizing the breast further comprises maintaining the breast within a customized breast mold.

40. (canceled)

41. The method of claim 33, where creating one or more 3D images of the breast further comprises:
receiving, by at least one processor, the one or more images taken at the one or more intervals; and
performing, by the at least one processor, a super-compounding process on the one or more images.

42. (canceled)

43. A method of treating breast tissue, the method comprising:
applying radiation to a treatment volume in a breast of a patient;
where the radiation is directed to the treatment volume in accordance with a 3D model generated from a plurality of ultrasound images of the breast; and
where the breast remains immobilized between the time at which the ultrasound images are obtained and the radiation is applied to the treatment volume.
